(19) United States
(12) Patent Application Publication
(10) Pub. No.: US 2006/0275232 A1
(43) Pub. Date: Dec. 7, 2006

(54) Two-composition product, uses thereof, and makeup kit containing this product

(75) Inventor: Veronique Chevalier, Villecresnes (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/437,677

(22) Filed: May 22, 2006

Related U.S. Application Data
(60) Provisional application No. 60/690,092, filed on Jun. 14, 2005.

(30) Foreign Application Priority Data
Jun. 1, 2005 (FR) 05 51451

(51) Int. Cl. A61K 8/49

(52) U.S. Cl. 424/63; 424/64; 424/70.12

(57) ABSTRACT

A product for making up and/or caring for the skin, the lips or the integuments, containing a first and a second composition, the first composition being a W/O emulsion containing at least one wax and fibres, the second composition containing a fatty phase with at least one oil. The product may constitute a foundation, a makeup rouge, an eye shadow, a concealer, a blusher, a lipstick, a lip balm, a lip gloss, a lip pencil, an eye pencil, a mascara, an eye liner, a skincare, body, makeup or skin colouring product. Process using this product. Makeup kit containing the product.

### REFERENCE TO PRIOR APPLICATIONS

This application claims priority to U.S. provisional application 60/690,092 filed Jun. 14, 2005, and to French patent application 0551451 filed Jun. 1, 2005, both incorporated herein by reference.

### FIELD OF THE INVENTION

The present invention relates to a novel product comprising at least two compositions that may be applied to the skin of the human face and/or body, to the lower and upper eyelids, to the lips or to the integuments (especially the eyelashes) of human beings, etc.

The product and one and/or the other of the compositions constituting the product may be a foundation, a makeup rouge, an eye shadow, a concealer product, a blusher, a lipstick, a lip balm, a lip gloss, a lip pencil, an eye pencil, a mascara, an eye liner, a skincare product, a body makeup product, a skin colouring product, etc.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In the cosmetics field, and especially in the field of makeup, the development of compositions endowed both with good properties in terms of application to and comfort on the skin, and satisfactory properties in terms of staying power, especially gloss staying power, and of migration resistance, remains a permanent objective. The raw materials generally used for gloss are very glossy viscous oils with a high refractive index, which are preferably hydrocarbon-based, for instance polybutenes. However, these raw materials may under certain conditions pose problems of tack and above all of migration when they are used in high content.

The substantial migration of a fatty phase containing oils and charged with dyestuffs or active products leads to an unattractive effect, which particularly accentuates wrinkles and fine lines, or makes the contour of the deposit more hazy. This migration is often mentioned by women as being a major defect of standard lipsticks.

It is known that it is possible to limit the migration of compositions by means of adding fillers. However, it has also been found that this addition of filler generally takes place to the detriment of the gloss of the compositions obtained and usually leads to dryness experienced during the application of the formulations.

Consequently, there is still a need to obtain glossy compositions endowed with an intense colour and satisfactory comfort, but without migration.

### SUMMARY OF THE INVENTION

The inventor has discovered a unique combination where a first composition is used to limit the migration of a second composition containing a fatty phase containing at least one oil.

The present invention makes it possible to overcome the drawbacks of the products of the prior art. The product of the invention makes it possible, for example, to limit the migration of the second composition beyond the lips or around the eyes. The selection of a particular base (first composition) makes it possible to obtain a cosmetic product, for example a makeup product, in particular a glossy and comfortable makeup product, which does not migrate, this being difficult to obtain in the past.

### DETAILED DESCRIPTION OF THE PREFERRED EMBODIMENTS

One subject of the invention is a cosmetic product for making up and/or caring for the skin, the lips and/or the integuments, comprising a first and a second composition,
the first composition comprising a W/O emulsion comprising an aqueous phase dispersed in an oily phase, and containing at least one wax and fibres,
the second composition comprising a fatty phase containing at least one oil.

By applying the first composition, the product of the invention can limit the migration of the second composition beyond the trace or the initial deposit of the first composition. The migration may be reflected by running of the composition in the wrinkles and fine lines at the periphery of the deposit. The migration may also be reflected by a continuous overflowing of the composition on the entire periphery of the deposit.

The first composition is thus preferably intended to fix the deposit of the second composition that would have a tendency to migrate beyond the initial trace if it were applied alone directly to, e.g., the skin or the lips.

The product of the invention is particularly suitable for making makeup products for the lips such as lipsticks or lip glosses, for the eyes, such as eye liners or eye shadows, or for facial skin, such as foundations or makeup rouges.

The term “makeup product” means a product containing a colouring agent (including skin-tone coloured agents) for depositing a colour on the skin or the lips, such as lipsticks, makeup powders, eye liners, foundations, self-tanning products or semi-permanent makeup products (tattoos).

The product according to the invention comprises two compositions. The two compositions may be packaged separately or together in the same packaging article or in two (or more) separate or distinct packaging articles. Preferably, these compositions are packaged separately and, advantageously, in separate packaging articles.

A subject of the invention is also a makeup kit containing a cosmetic makeup or care product as defined above, in which the two compositions are packaged separately and are accompanied by one or more suitable application means. These means may be fine brushes, coarse brushes, pens, pencils, felts, feathers, sponges and/or foams.

The first composition of the product according to the invention typically constitutes a base coat applied to the skin, the lips or the integuments, and the second composition constitutes a top coat, but this is not required.

The invention also relates to a process for making up the skin, the lips and/or the integuments, which comprises successively applying to the skin, the lips and/or the integuments the first and second compositions of the cosmetic makeup product as defined above. The second composition is preferably a makeup composition containing pigments or dyes. The order of application is preferably first composition first, second composition thereafter, preferably with the second composition applied such that it at least partially overlaps the first composition. In preferred embodiments the second composition is applied such that it substantially overlaps the first composition or is within the boundaries of the first composition as applied.

The invention also relates to a process for making up or caring for the skin and/or the lips and/or the integuments, which comprises applying to the skin, the lips and/or the integuments a first coat of the said first composition described above, and then in applying at least partially onto this first coat a second coat of the second composition described above.

The first and second coats, and particularly the second coat, may form patterns and may be applied with a pen, a pencil or any other instrument (sponge, finger, fine brush, coarse brush, foam, feather, etc.). This makeup may also be applied onto makeup accessories, for instance false nails, false eyelashes or wigs, or alternatively spots or patches adhering to the skin or the lips (of the beauty-spot type).

A subject of the invention is also the use of a composition in the form of a W/O emulsion comprising at least one wax and fibres, for reducing the migration of a cosmetic composition comprising a fatty phase containing at least one oil.

A subject of the invention is also the use, for caring for or making up the lips, of a composition in the form of a W/O emulsion comprising at least one wax and fibres, for limiting the migration of another composition comprising a fatty phase containing at least one oil.

I. First Composition

The first composition preferably constitutes the base once applied and is preferably used as the first coat. It is in the form of a W/O emulsion comprising an aqueous phase dispersed in an oily phase, this dispersion preferably being prepared using an emulsifying surfactant, preferably a silicone surfactant.

Since this W/O emulsion is intended for topical application, it preferably comprises a physiologically acceptable medium. The term “physiologically acceptable medium” means a medium that is compatible with keratin materials such as the skin, the lips, the nails, the scalp and/or the hair.

The W/O emulsion contains fibres, and at least one wax. The wax is preferably located in the oily phase. According to one preferred embodiment of the invention, the oily phase of the W/O emulsion is in the form of a paste that is soft at room temperature.

The term “room temperature” means herein a temperature ranging from about 20 to 25° C.

The term “soft paste” means a paste whose viscosity can be measured, as opposed to the solid structure of a wand or stick, whose viscosity cannot be measured. The dynamic viscosity of the soft paste at 25° C. is preferably between 3 and 35 Pa·s, measured using a Contraves TV rotary viscometer equipped with an “MS-r4” spindle at a frequency of 60 Hz. Usually, the oily phase containing waxes is prepared with heating and, when it is cooled, it resolidifies, and as such its mixing with the aqueous phase should also be performed with heating to avoid this resolidification, whereas, when the oily phase is in the form of a soft paste at room temperature, this means that it does not resolidify and that it can be mixed with the aqueous phase at room temperature.

This soft paste can be obtained, for example, by heating the waxes and other fatty substances (oils and other fatty substances) of the oily phase (except for the emulsifying surfactant, which is often added later), followed by blending of the mixture while cooling it to room temperature. It is also possible to first melt the waxes and solid fatty substances, and then to add thereto the oils and other constituents of the oily phase and to blend the mixture while cooling it, the important point for obtaining a soft paste being the cooling during the blending.

According to a more particularly preferred embodiment of the invention, the oily phase and/or the W/O emulsion are prepared using a blender such as a mixer-extruder.

The W/O emulsion constituting the first composition of the invention is preferably in the form of a cream, i.e. a soft product as opposed to a solid product such as a stick. A cream has a viscosity at room temperature (about 20 to 25° C.) preferably ranging from about 10 to 250 poises (1 to 25 Pa·s) and more preferably ranging from about 10 to 100 poises (1 to 10 Pa·s), this viscosity being measured with a Rheomat 180 viscometer with a suitable spindle (for example a No. 2, 3 or 4 spindle).

When the oily phase of the W/O emulsion is in the form of a soft paste, the W/O emulsion obtained, even when it contains a large amount of wax, has the advantage of being able to contain a large amount of aqueous phase. As a result, it has the advantage of feeling fresh on application to the topical support (skin, lips or integuments). In addition, the mixing of the aqueous phase and of the oily phase may be performed without heating, and heat-sensitive compounds may thus be incorporated without any fear of them being degraded.

Fibres

The fibres may be present in the W/O emulsion of the invention in an amount ranging for example from 0.1% to 30% by weight, preferably from 1% to 20% by weight, better still from 3% to 15% by weight and even better still from 5% to 10% by weight relative to the total weight of the composition.

The fibres are preferably introduced into the hydrophobic (or oily) phase.

The term “fibre” should be understood as meaning an object of length L and diameter D such that L is greater than D and preferably very much greater than D, D being the diameter of the circle in which the cross section of the fibre is inscribed. In particular, the ratio L/D (or shape factor) is preferably chosen in the range from 3.5 to 2500, preferably from 5 to 500 and in particular from 5 to 150.

The fibres that may be used in the composition of the invention are not particularly limited and may be mineral or organic fibres of synthetic or natural origin and they may be soft or solid. They may be short or long, individual or organized, for example braided. They may have any shape, and may especially have a circular or polygonal (square, hexagonal or octagonal) cross section, depending on the intended specific application. In particular, their ends are preferably blunt and/or polished to prevent injury.

In particular, the fibres preferably have a length ranging from 1 μm to 10 mm, more preferably from 0.1 mm to 5 mm and better still from 0.1 mm to 3 mm. They preferably have a cross section that is within a circle of diameter ranging from 2 nm to 500 μm, and more preferably ranging from 100 nm to 100 μm. The weight of the fibres is often given in denier or decitex.

The fibres useful herein include those used in the manufacture of textiles, and in particular silk fibre, cotton fibre, wool fibre, flax fibre, cellulose fibre extracted for example from wood, from plants or from algae, polyamide fibre (Nylon®, in particular under the names Nylon 6=Polyamide 6; Nylon 6,6 or Nylon 66=Polyamide 6,6; Nylon 12=Polyamide 12), rayon fibre, viscose fibre, acetate fibre, in particular rayon acetate fibre, cellulose acetate fibre or silk acetate fibre, poly(p-phenyleneterephthalamide), acrylic polymer fibre, in particular polymethyl methacrylate fibre or poly(2-hydroxyethyl methacrylate) fibre, polyolefin fibre and in particular polyethylene or polypropylene fibre, glass fibre, silica fibre, carbon fibre, in particular in graphite form, polytetrafluoroethylene (such as Teflon®) fibre, insoluble collagen fibre, polyester fibre, polyvinyl chloride fibre or polyvinylidene chloride fibre, polyvinyl alcohol fibre, polyacrylonitrile fibre, chitosan fibre, polyurethane fibre, polyethylene phthalate fibre, and fibres formed from a mixture of polymers such as those mentioned above, for instance polyamide/polyester fibres, and mixtures of these fibres.

The fibres used in surgery may also be used, for instance the resorbable synthetic fibres prepared from glycolic acid and caprolactone (Monocryl from Johnson & Johnson); resorbable synthetic fibres of the type which is a copolymer of lactic acid and of glycolic acid (Vicryl from Johnson & Johnson); polyterephthalic ester fibres (Ethibond from Johnson & Johnson) and stainless steel threads (Acier from Johnson & Johnson).

Moreover, the fibres may be treated or untreated at the surface, and coated or uncoated. As coated fibres that may be used in the invention, mention may be made of polyamide fibres coated with copper sulfide to give an anti-static effect (for example R-STAT fibres from the company Rhodia) or fibres coated with another polymer enabling a particular organization of the fibres (specific surface treatment) or surface treatment inducing colour/hologram effects (for example the “Lurex” fibre from the company Sildorex).

The fibres that may be used in the composition according to the invention are preferably chosen from polyamide fibres, cellulose fibres and polyethylene fibres, and mixtures thereof. They may range for example from 0.1 to 5 mm and preferably from 0.25 to 1.6 mm in length, and their mean diameter may range from 5 to 50 μm.

According to one preferred embodiment of the invention, the fibres are chosen from Nylon 6 (or Polyamide 6), Nylon 6,6 or Nylon 66 (or Polyamide 6,6) and Nylon 12 (or Polyamide 12) fibres, and mixtures thereof.

In particular, it is possible to use the polyamide fibres sold by Etablissements P. Bonte under the name Polyamide 0.9 dtex 0.3 mm (INCI name: Nylon 6,6), having a mean diameter of 6 μm, a weight of about 0.9 dtex and a length ranging from 0.3 mm to 3 mm, or alternatively the polyamide fibres sold under the name Fiberlon 931-D1-S by the company LCW, having a yarn count of about 0.9 dtex and a length of about 0.3 mm. It is also possible to use Nylon-66 fibres, having a yarn count of about 2 dtex and a length of about 0.3 mm, sold under the name “Polyamide brillante trilobée” by the company Utexbel (INCI name: Nylon-66).

It is also possible to use cellulose (or rayon) fibres for example with a mean diameter of 50 μm and a length ranging from 0.5 mm to 6 mm, for instance those sold under the name “Natural rayon flock fiber RC1BE-N003-M04” by the company Claremont Flock. It is also possible to use polyethylene fibres, for instance those sold under the name “Shurt Stuff 13 099 F” by the company Mini Fibers.

Waxes

The W/O emulsion of the invention contains one or more waxes, preferably present in the oily phase. The amount of wax(es) in the W/O emulsion of the invention is preferably at least 4% by weight and better still at least 5% by weight relative to the total weight of the emulsion. It may range, for example, from 4% to 30%, preferably from 5% to 30% and better still from 5% to 15% by weight relative to the total weight of the emulsion.

As waxes that may be used in the W/O emulsion of the invention, examples that may be mentioned include mineral waxes such as microcrystalline waxes, paraffin, petrolatum, petroleum jelly, ozokerite and montan wax; waxes of animal origin such as beeswax, lanolin and derivatives thereof; waxes of plant origin such as candelilla wax, ouricury wax, carnauba wax, Japan wax, cocoa butter, cork fibre wax or sugarcane wax; hydrogenated oils that are solid at 25° C.; fatty esters and glycerides that are solid at 25° C.; synthetic waxes such as polyethylene waxes and the waxes obtained by Fischer-Tropsch synthesis; silicone waxes, and mixtures thereof.

According to one preferred embodiment of the invention, at least one wax with a starting melting point of greater than or equal to 50° C., and better still at least one wax whose starting melting point is greater than or equal to 65° C., is used, such as carnauba wax, polyethylene waxes with a starting melting point of greater than 50° C. and better still greater than 65° C., and microcrystalline waxes with a starting melting point of greater than 50° C. and better still 65° C., such as the product sold by the company Tisco under the name Tisco Wax 88 or the product sold by the company RMC under the name Feruwax 30540, and mixtures thereof.

In the present description the term “starting melting point” means the temperature at which a wax begins to melt. This temperature may be determined by DTA (differential thermal analysis), which allows the thermogram (or melting curve) of the wax under consideration to be obtained. The starting melting point corresponds to the temperature at which an appreciable change in the slope of the thermogram may be observed. The melting point itself represents the minimum point of the said thermogram.

Emulsifying Surfactant

According to one preferred embodiment of the invention, the W/O emulsion contains at least one emulsifying surfactant. This surfactant is preferably a silicone surfactant. The amount (as active material) of emulsifying surfactant(s) in the W/O emulsion preferably ranges from 0.02% to 5% by weight of active material, better still from 0.05% to 4% by weight and even better still from 0.1% to 2% by weight of active material relative to the total weight of the composition.

The silicone emulsifying surfactant may be chosen especially from the group comprising oxyethylenated and/or oxypropylenated polydimethylsiloxanes, also known as dimethicone copolyols, oxyethylenated and/or oxypropylenated C₁₀-C₂₂-alkyl-polydimethylsiloxanes, also known as alkyldimethicone copolyols, and oxyethylenated and/or oxypropylenated polydimethylsiloxanes containing glucoside groups, and mixtures thereof.

Examples of dimethicone copolyols that may be mentioned include the mixture of dimethicone copolyol, of cyclopentasiloxane (cyclomethicone) and of water (10/88/2), sold by the company Dow Corning under the name DC3225C or DC-5225C (INCI name: cyclopentasiloxane/PEG/PPG-18/18 dimethicone), and the mixture of dimethicone copolyol and of cyclopentasiloxane (85/15) sold under the name Abil EM-97 by the company Goldschmidt (INCI name: BIS-PEG/PPG-14/14 dimethicone/cyclopentasiloxane).

Alkyldimethicone copolyols that may especially be mentioned include those with an alkyl radical containing from 10 to 22 carbon atoms, such as cetyl dimethicone copolyol, for instance the product sold under the name Abil EM-90 by the company Goldschmidt; lauryl dimethicone copolyol, for example the mixture of about 91% lauryl dimethicone copolyol and of about 9% isostearyl alcohol, sold under the name Q2-5200 by the company Dow Corning, and mixtures of these silicone surfactants.

Polydimethylsiloxanes containing glucoside groups that may especially be mentioned include the compounds of formula (I) 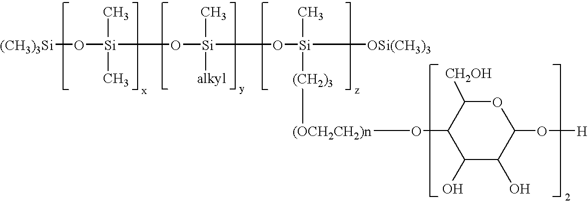
in which:

x, y and z each represent a positive integer ranging from 1 to 10 000, preferably from 1 to 5000 and better still from 1 to 2000,

n represents a positive integer ranging from 1 to 20 and preferably from 1 to 15,

the alkyl radical represents a saturated, linear or branched hydrocarbon-based radical containing from 2 to 20 and preferably from 2 to 8 carbon atoms,

and particularly the compound of formula (I) in which the alkyl radical is an n-octyl radical, sold in the form of a liquid solution containing 15% active material in a cyclomethicone under the name SPG 128 VP by the company Wacker.

The silicone surfactant is preferably a dimethicone copolyol, more particularly the product sold under the name DC3225C or DC5225C.

Oily Phase

The oily phase of the W/O emulsion of the invention may contain, besides the waxes indicated above, oils and/or fatty substances of any kind including those that are well known to those skilled in the art, for instance oils of plant origin (for example jojoba oil, avocado oil, sesame oil, sunflower oil, corn oil, soybean oil, safflower or grapeseed oil), mineral oils (for instance petroleum jelly or optionally hydrogenated isoparaffins), synthetic oils (for example isopropyl myristate, cetearyl octanoate, polyisobutylene, ethylhexyl palmitate or alkylbenzoates), volatile or non-volatile silicone oils, and fluoro oils or fluorosilicone oils, and also mixtures of these oils.

Preferably, the oily phase of the composition of the invention comprises at least one silicone oil, which may be present in an amount ranging, for example, from 3% to 50% by weight, preferably from 5% to 30% by weight and better still from 9% to 30% by weight relative to the total weight of the composition. Examples of silicone oils that may be mentioned include volatile silicone oils such as cyclodimethylsiloxanes or cyclomethicones, for instance pentacyclomethicone, tetracyclomethicone or hexacyclomethicone; non-volatile silicone oils such as polydimethylsiloxanes (PDMS). The silicone oil may be the oil contained in the raw material constituting the silicone surfactant, as is the case when DC-5225C or DC-3225C is used.

The oily phase may also contain other fatty constituents such as fatty alcohols, for instance stearyl alcohol, cetyl alcohol and cetearyl alcohol, fatty acids, gums, for example silicone gums, for instance the mixture of PDMS containing α,ω-hydroxylated groups/PDMS 5 cSt (12/88) sold under the name DC 1503 by the company Dow Corning, and lipophilic gelling agents such as bentone.

The oily phase may also contain lipophilic adjuvants.

The oily phase may be present in the emulsion in an amount ranging for example from 15% to 70% by weight and preferably from 25% to 60% by weight relative to the total weight of the emulsion, this amount comprising the amount of emulsifying surfactant.

As indicated above, the cooled oily phase is advantageously, before being mixed with the aqueous phase, in the form of a soft paste, i.e. the soft paste having a dynamic viscosity at 25° C. generally of between 3 and 35 Pa·s, measured using a Contraves TV rotary viscometer equipped with an “MS-r4” spindle at a frequency of 60 Hz.

This soft paste may be obtained especially by heating the waxes and other solid substances of the oily phase, followed by blending the mixture of this molten premix with the other constituents of the oily phase while cooling the mixture to room temperature.

Aqueous Phase

The aqueous phase of the W/O emulsion of the invention may range for example from 30% to 85% by weight and preferably from 40% to 75% by weight relative to the total weight of the emulsion. It contains water, and may contain, besides water, solvents and hydrophilic adjuvants. Examples of solvents that may be mentioned include primary alcohols containing from 1 to 4 carbon atoms, for instance ethanol, or polyols, for instance butylene glycol, glycerol and hexylene glycol. The solvent(s) may be present in an amount ranging for example from 0.1% to 30% by weight relative to the total weight of the emulsion.

The W/O emulsion may also comprise one or more adjuvants as described below.

One particular mode of preparation of the W/O emulsion of the invention comprises performing the preparation of the oily phase and optionally of any emulsion using a mixer-screw extruder, subjected to a temperature gradient ranging from 100° C. to 10° C., and in introducing the fibres first into the mixer-extruder.

The soft paste may be obtained, for example, by melting the waxes and other solid fatty substances and blending this molten premix with the oils and the other constituents of the oily phase, this blending being performed while cooling this mixture to room temperature, it especially being possible for the blending to be performed in a mixer-extruder.

The process for preparing the emulsion in a mixer-extruder may more specifically include the following steps:
(1) preparation of the oily phase by mixing together, with heating, the waxes, oils and other lipophilic constituents of the oily phase, between 80° C. and 100° C.,
(2) introduction of the fillers and fibres into the first sheath of the mixer-extruder;
(3) introduction of the molten mixture obtained in (1) into the second sheath heated to the temperature at which the mixture of (1) was prepared;
(4) blending of the mixture of (2) and (3) while cooling to room temperature by passing through several sheaths and production of a soft paste;
(5) incorporation of the emulsifying surfactant into the soft paste obtained in (4), and
(6) incorporation, with stirring, of the aqueous phase and optionally of fillers not introduced in (2) into the mixture obtained in (5).

Another process comprises introducing into the same sheath the molten mixture of the waxes and oils and the fillers and fibres.

The use of a mixer-extruder makes it possible to reproducibly obtain a paste with an oily phase of very consistent quality. Furthermore, it is possible, by adapting the output die of the mixer-extruder, to condition the W/O emulsion on-line at the outlet of the said mixer-extruder.

The various steps of the process may be performed in one or more extruders arranged one after the other, and preferably in a single twin-screw extruder.

The conditions under which the extrusion may be performed are described in document FR-A-2 715 306, the content of which is incorporated into the present patent application by reference.

The length of the sheaths may vary. It is preferably 100 mm, and additional sheaths may be added if necessary for the separate introduction of other compounds.

II. Second Composition

The second composition comprises a fatty phase containing at least one oil. This composition preferably constitutes the upper coat of the product and is applied over the base preferably constituted by the first composition.

For the purposes of this patent application, the term “oil” means any non-aqueous medium that is liquid at room temperature (20-25° C.) and at atmospheric pressure (760 mm Hg).

The second composition may consist solely of the fatty phase. It may also comprise another phase, for example an aqueous phase.

The fatty phase comprises the oils and the other fatty substances such as waxes, gums and pasty substances.

The amount of oils in the second composition preferably represents at least 20% by weight and better still at least 30% by weight relative to the total weight of this composition. This amount may range, for example, from 20% to 90% and better still, preferably, from 30% to 85% of the total weight of this composition.

According to one preferred embodiment of the invention, the second composition contains at least one non-volatile hydrocarbon-based oil.

As non-volatile hydrocarbon-based oils that may be used in the invention, mention may be made of:
linear or branched hydrocarbons such as liquid paraffin, liquid petroleum jelly, light naphthalene oil and hydrogenated polyisobutene (Parleam oil);
hydrocarbon-based oils of animal origin, for instance squalene and perhydrosqualene (or squalane);
liquid triglycerides of fatty acids containing at least 10 carbon atoms;
synthetic esters and ethers especially of fatty acids, for instance the oils of formula R₁(CO)ₓOR₂ in which R₁ represents an acid residue containing from 2 to 29 carbon atoms with x being 0 or 1 and R₂ represents a hydrocarbon-based chain containing from 3 to 30 carbon atoms, for instance tributyl acetyl citrate, oleyl erucate, 2-octyldodecyl behenate, triisoarachidyl citrate, isocetyl or octyldodecanyl stearoylstearate, n-propyl acetate, tridecyl trimellitate, diisocetyl dodecanedioleate or stearate, arachidyl propionate, dibutyl phthalate, propylene carbonate, octyldodecyl pentanoate; polyol esters, for instance vitamin F, sorbitan isostearate, glyceryl or diglyceryl triisostearate and polyglyceryl-2 diisostearate; diisostearyl malate, isopropyl palmitate, diisopropyl adipate, caprylic/capric acid triglycerides, for instance those sold by the company Stearineries Dubois or those sold under the names Miglyol 810, 812 and 818 by the company Dynamit Nobel, shea butter oil, isopropyl myristate, butyl stearate, hexyl laurate, diisopropyl adipate, isononyl isononate, 2-hexyldecyl laurate, 2-octyldecyl palmitate, 2-octyldodecyl myristate or lactate, 2-diethylhexyl succinate, 2-ethylhexyl palmitate, 2-octyldodecyl stearate and castor oil; diisopropyl dimerate (INCI name: diisopropyl dimer dilinoleate) such as the product sold under the name Schercemol DID by the company Noveon;
fluoro oils;
fatty alcohols containing from 7 to 29 carbon atoms, such as stearyl alcohol, linoleyl alcohol, linolenyl alcohol, isostearyl alcohol, 2-octyldodecanol, decanol, dodecanol, octadecanol or oleyl alcohol;
fatty acids containing from 7 to 29 carbon atoms, such as myristic acid, palmitic acid, stearic acid, behenic acid, oleic acid, linoleic acid, linolenic acid or isostearic acid;
mixtures thereof.

The second composition may also contain silicone oils, preferably non-volatile silicone oils. Non-volatile silicone oils that may be mentioned include polydimethylsiloxanes (PDMS), optionally comprising a C₃-C₄₀ alkyl or alkoxy chain or a phenyl chain such as phenyl trimethicones, polyalkylmethylsiloxanes, which are optionally fluorinated, for instance polymethyltrifluoropropyldimethylsiloxanes, or which optionally contain functional groups such as hydroxyl, thiol and/or amine groups; polysiloxanes modified with fatty acids, fatty alcohols or polyoxyalkylenes.

The second composition may also comprise one or more waxes and/or one or more pasty substances, in particular when it is in the form of a stick.

For the purposes of the present invention, the term “pasty substance” means a lipophilic fatty compound with a reversible solid/liquid change of state, which has an anisotropic crystal organization in the solid state, and which comprises a liquid fraction and a solid fraction at 23° C.

The pasty compound is advantageously chosen from:
lanolin and its derivatives,
polymeric or non-polymeric silicone compounds,
polymeric or non-polymeric fluoro compounds,
vinyl polymers, especially:
   olefin homopolymers,
   olefin copolymers such as polyvinylpyrrolidone/α-olefin copolymers, for instance the copolymer of vinylpyrrolidone and of hexadecene sold under the name Antaron V216 by the company ISP,
   hydrogenated diene homopolymers and copolymers,
   linear or branched homopolymeric or copolymeric oligomers of alkyl (meth)acrylates preferably containing a C₈-C₃₀ alkyl group,
   homopolymeric and copolymeric oligomers of vinyl esters containing C₈-C₃₀ alkyl groups,
   homopolymeric and copolymeric oligomers of vinyl ethers containing C₈-C₃₀ alkyl groups,
liposoluble polyethers resulting from polyetherification between one or more C2-C100 and preferably C2-C50 diols,
esters, for instance the mixture of glyceryl esters of isostearic/adipic fatty acids,
   and mixtures thereof.

The pasty compound may represent, for example, from 1% to 99%, preferably from 1% to 60%, better still from 2% to 30% and even better still from 5% to 15% by weight of the composition constituting the second composition of the product according to the invention.

The waxes may be the same as those used for the first composition. Among these waxes, mention may be made especially of waxes of animal, plant, mineral or synthetic origin, such as liquid jojoba wax, microcrystalline waxes, paraffin, petroleum jelly, ozokerite or montan wax; beeswax, lanolin and its derivatives; candelilla wax, ouricury wax, carnauba wax, Japan wax, cocoa butter, cork fibre wax or sugarcane wax; hydrogenated oils that are solid at 25° C., ozokerites, fatty esters and glycerides that are solid at 25° C.; polyethylene waxes and the waxes obtained by Fischer-Tropsch synthesis; hydrogenated oils that are solid at 25° C.; lanolins; fatty esters that are solid at 25° C.; silicone waxes; fluoro waxes; mixtures thereof.

The second composition of the product according to the invention may be in any galenical form, including those normally used for topical application and especially in the form of an oily or aqueous solution, an oily or aqueous gel, an oil-in-water or water-in-oil emulsion, a multiple emulsion, a dispersion of oil in water by means of vesicles, the vesicles being located at the oil/water interface, or a powder. It may be fluid or solid. It may have the appearance of a lotion, a cream, a pomade, a soft paste, an ointment, a cast or moulded solid especially as a stick or in a dish, or alternatively a compacted solid.

According to one preferred embodiment of the invention, it is in the form of a cast or moulded solid, especially in the form of a lipstick in stick form, a lip gloss, a cast foundation or a cast blusher. It may also be, for example, a liquid lip gloss.

Advantageously, the second composition has a continuous fatty phase and is preferably in anhydrous form. The term “anhydrous” means that the composition may contain less than 5% water and better still less than 1% water relative to the total weight of the second composition.

The second composition may be prepared using techniques known to a person skilled in the art. It may be obtained especially by heating the various constituents to the melting point of the highest-melting waxes, followed by pouring the molten mixture into a mould (dish or finger stall). It may also be obtained by extrusion as described in patent application EP-A-0 667 146.

Adjuvants

Each of the two compositions of the invention may also contain adjuvants such as those that are common in cosmetics and/or dermatology, such as lipophilic or hydrophilic gelling agents, active agents, preserving agents, antioxidants, complexing agents, pH regulators (acidic or basic), fragrances, fillers, bactericides, odour absorbers, dyes and lipid vesicles. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

Thus, the compositions of the invention may especially contain lipophilic gelling agents such as bentones and hectorites, and such as elastomeric organopolysiloxanes, for instance those sold under the names KSG 6 from Shin-Etsu, Trefil E-505C or Trefil E-506C from Dow Corning, Gransil (SR-CYC, SR DMF 10 or SR-DC556) from Grant Industries, or those sold in the form of ready-constituted gels: KSG 15, KSG 17, KSG 16 and KSG 18 from Shin-Etsu, Gransil SR 5CYC Gel, Gransil SR DMF 10 Gel and Gransil SR DC556 Gel from Grant Industries, and 1229-02-167 and 1229-02-168 from General Electric. A mixture of these compounds may also be used.

Among the fillers that may be used in the compositions of the invention, examples that may be mentioned include pulverulent dyestuffs; silica powder; talc; mica; kaolin; polyamide particles and especially those sold under the name Orgasol by the company Atochem; polyethylene powders; microspheres based on acrylic copolymers, such as those made of ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or non-crosslinked corn starch, wheat starch or rice starch, such as powders of starch crosslinked with octenylsuccinate anhydride, sold under the name Dry-Flo by the company National Starch; silicone resin microbeads such as those sold under the name Tospearl by the company Toshiba Silicone; and mixtures thereof. These fillers may be present in amounts ranging from 0 to 20% by weight and preferably from 1% to 10% by weight relative to the total weight of the emulsion.

Pulverulent dyestuffs that may be mentioned in particular include pigments, nacres and flakes, and mixtures thereof.

The pigments may be white or coloured, mineral and/or organic, and interference or non-interference pigments. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue. Among the organic pigments that may be mentioned are carbon black, pigments of the type such as organic lakes of barium, strontium, calcium or aluminium, including those submitted for certification by the Food and Drug Administration (FDA) (for example D&C or FD&C) and those that are exempt from FDA certification, for instance lakes based on cochineal carmine. The pigments may represent for example from 0.1% to 50%, especially from 0.5% to 35% and better still from 2% to 25% by weight of active material relative to the total weight of the composition.

The nacreous pigments may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride. They may represent from 0 to 25% (as active material) and better still from 0.1% to 15% of the total weight of the composition (if present). Pigments with goniochromatic properties and/or pigments with a metallic effect, as described in the patent application filed for the number FR-A-2 842 417, the content of which is incorporated into the present patent application by reference, may thus be used.

According to one preferred embodiment of the invention, the first composition contains less than 10% by weight, preferably less than 5%, more preferably less than 2% and even more preferably less than 1% by weight of pigments, whereas the second composition contains at least 0.1% of pigments, preferably at least 1% of pigments, more preferably at least 5% by weight of pigments and even more preferably at least 10% by weight of pigments.

As active agents that may be used in the compositions of the invention, examples that may be mentioned include enzymes (for example lactoperoxidase, lipase, protease, phospholipase or cellulases); flavonoids; moisturizers such as protein hydrolysates; sodium hyaluronate; polyols, for instance glycerol, glycols, for instance polyethylene glycols, and sugar derivatives; anti-inflammatory agents; procyannidol oligomers; vitamins, for instance vitamin A (retinol), vitamin E (tocopherol), vitamin C (ascorbic acid), vitamin B5 (panthenol), vitamin B3 (niacinamide), derivatives of these vitamins (especially esters) and mixtures thereof; urea; caffeine; depigmenting agents such as kojic acid, hydroquinone and caffeic acid; salicylic acid and its derivatives; α-hydroxy acids such as lactic acid and glycolic acid and derivatives thereof; retinoids such as carotenoids and vitamin A derivatives; sunscreens; hydrocortisone; melatonin; algal, fungal, plant, yeast or bacterial extracts; steroids; antibacterial active agents, for instance 2,4,4′-trichloro-2′-hydroxydiphenyl ether (or triclosan), 3,4,4′-trichlorocarbanilide (or triclocarban) and the acids indicated above and especially salicylic acid and its derivatives; matting agents; tensioning agents; ceramides; essential oils; and mixtures thereof; and any active agent that is suitable for the final purpose of the composition.

The active agent(s) may be present, for example, in a concentration ranging from 0.01% to 20%, preferably from 0.1% to 5% and better still from 0.5% to 3% of the total weight of the composition.

Needless to say, a person skilled in the art will take care to select the optional compound(s) to be added to the compositions of the invention such that the advantageous properties intrinsically associated with the product in accordance with the invention are not, or are not substantially, adversely affected by the envisaged addition.

III. Product According to the Invention

This product comprises the two compositions described above. The product according to the invention may be advantageously used for making up or caring for the skin and/or the lips and/or the integuments according to the nature of the ingredients used. In particular, it may constitute a foundation, a makeup rouge, an eye shadow, a concealer product, a blusher, a lipstick, a lip balm, a lip gloss, a lip pencil, an eye pencil, a mascara, an eye liner, a skincare product, a body makeup product or a skin colouring product. The product is in particular a lipstick.

Each composition may be packaged separately, and they may be packaged separately in the same packaging article, for example in a two-compartment pen, the base compartment being delivered from one end of the pen and the top composition being delivered from the other end of the pen, each end being closed especially in a leaktight manner by means of a cap.

It is also possible for each composition to be present separately, for example in two tubes or two jars or a tube and a jar.

The composition that is applied as the first coat is preferably in the form of a cream, which allows a more practical application.

Advantageously, the second composition, when used as top coat, has care and/or gloss and/or colouring properties.

A subject of the invention is also a lip product, a foundation, a tattoo, a makeup rouge or an eye shadow containing a first and a second composition as described above.

The example below of compositions according to the invention is given by way of illustration and with no limiting nature. The names given therein are chemical names, INCI names or trade names. The amounts therein are given as weight percentages, unless otherwise mentioned.

### EXAMPLE 1

I. Base as first composition: W/O emulsion

| | |
|---|---|
| A. Oily phase | |
| Dry-Flo (filler) | 7% |
| Microcrystalline wax | 19% |
| Polyamide fibres (Nylon 6,6) | 19% |
| Mineral oil | qs 100% |
| W/O emulsion | |
| Oily phase | 40% |
| Cyclopentasiloxane/PEG/PPG- | 10% |
| 18/18 dimethicone (DC-5225C) | |
| Silica (filler) | 4% |
| Glycerol | 5% |
| Water | qs 100% |

Procedure 1:
the mixture of wax and oil was heated to about 100° C.,
this molten mixture was introduced into a mixer-extruder to the same level as the mixture of filler (Dry-Flo) and mineral oil, and the fibres were introduced via a weight meter. The oily phase in the form of a soft paste was obtained at the mixer-extruder outlet,
the silicone emulsifier was incorporated into the soft paste, in a rotor-stator,
the water, glycerol and silica were gradually added to the mixture with stirring.

Procedure 2:
the mixture of wax and oil was heated to about 100° C.,
the filler (Dry-Flo) and the fibres were introduced into the first sheath of a six-sheath mixer-extruder,
the molten mixture of wax and oil was introduced into the second element of the said mixer-screw extruder, and
the aqueous phase, the silicone emulsifier and the silica were introduced via two different entries, into the fourth element of the said mixer-screw extruder.

The elements of the mixer-twin-screw extruder used were, from the first to the sixth element, respectively maintained at the following temperatures: 100° C., 80° C., 60° C., 20° C., 20° C. and 20° C.

The emulsion obtained was creamy, fluid and non-greasy, and it had a powdery finish. It constituted a good base.

II. Second composition: anhydrous composition

| | |
|---|---|
| Liquid jojoba wax | 5.7% |
| Propylene carbonate | 0.03% |
| Shea butter | 3.6% |
| Vinylpyrrolidone/hexadecene copolymer | 2.4% |
| (Antaron V 216) | |
| Polyethylene wax (MW: 500) | 6.6% |
| Diisopropyl dimerate (name INCI: | qs 100% |
| Diisopropyl dimer dilinoleate) | |
| Glyceryl esters of isostearic/adipic | 6.1% |
| fatty acids | |
| Mica-titanium oxide | 2.2% |
| Calcium salt of lithol red | 2.5% |
| Hydrogenated isoparaffin | 7.1% |
| Black iron oxide | 0.1% |
| Brown, yellow iron oxides | 1.9% |
| Distearyldimethylammonium-modified | 0.5% |
| hectorite | |
| Di-tert-butyl-4-hydroxytoluene | 0.03% |
| Perhydrosqualene | 5.7% |
| Mixture of isopropyl, isobutyl and | 0.5% |
| n-butyl p-hydroxybenzoate (40/30/30) | |
| Ozokerite (wax of melting point: 74-77° C.) | 4.9% |
| Hydrogenated polyisobutene (Parleam oil) | 7.3% |
| Aluminium lake | 5% |
| Polyglyceryl-2 diisostearate | 9.7% |
| 2-octyldodecanol | 6.3% |

### EXAMPLE 2

This was identical to Example 1, except that the base composition (W/O emulsion) also contains 2% bentone (oily gelling agent). In the procedure, the bentone was introduced into the mixture of wax, oil and Dry-Flo.

The composition had good emollient qualities.

Tests of Efficacy as a Lipstick Base (LS)

1) Aesthetic Flash

Objective: to evaluate the influence of the base composition of Example 1 on the smoothness and the run-proof nature of a standard lipstick (LS) on 8 women with fine lines around the contour of the lips. The W/O emulsion used according to the invention was first applied as base composition, followed by a lipstick known to be particularly runny.

Result: 7 out of 8 users found that the base composition according to the invention had very good performance qualities as regards the staying power of the LS, especially on account of its migration resistance and its effect on holding the film (coverage and gloss).

2) Standard Instrumental Screening

Objective: to evaluate the colour fastness, the gloss and the degree of migration by using as base composition the W/O emulsion of Example 1, combined with a standard lipstick, on 6 women.

Result: under the experimental conditions of applying the composition according to the invention as a base followed by a standard LS, no migration of the lipstick was observed one hour after application, nor was any significant difference in gloss observed between the gloss on application and that after one hour. Similarly, the colour fastness one hour after application remained identical to that obtained during application.

Another test confirmed that the base compositions used according to the invention are excellent makeup bases and provide an increase in the staying power of a foundation over time.

3) Care Cabin Flash Test

Objective: to evaluate the cosmetic aspects of the base composition of Example 1 of the invention, and also the uniformity of a standard foundation and a standard lipstick, applied onto this base composition, on 7 women.

The foundation or the lipstick were easy to apply, the application was uniform and the applied products adhered well. The makeup result is uniform.

The above written description of the invention provides a manner and process of making and using it such that any person skilled in this art is enabled to make and use the same, this enablement being provided in particular for the subject matter of the appended claims, which make up a part of the original description and including a cosmetic product for making up and/or caring for the skin, the lips or the integuments, containing a first and a second composition, the first composition consisting of a W/O emulsion comprising an aqueous phase dispersed in an oily phase, and containing at least one wax and fibres, and the second composition comprising a fatty phase containing at least one oil.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A product comprising a first and a second composition,
the first composition being a W/O emulsion comprising an aqueous phase dispersed in an oily phase, said W/O emulsion comprising at least one wax and fibres,
the second composition comprising a fatty phase which comprises at least one oil.

2. The product according to claim 1, wherein the fibres have a length ranging from 1 μm to 10 mm.

3. The product according to claim 1, wherein the fibres have a cross section within a circle of diameter ranging from 2 nm to 500 μm.

4. The product according to claim 1, wherein the fibres are present in an amount of 0.1% to 30% by weight relative to the total weight of the W/O emulsion.

5. The product according to claim 1, wherein the fibres are chosen from silk, cotton, wool, flax, cellulose, rayon, polyamide, viscose, acetate, poly-p-phenyleneterephthalamide, acrylic polymer, polyolefin, glass, silica, carbon, polytetrafluoroethylene, insoluble collagen, polyester, polyvinyl chloride, polyvinylidene chloride, polyvinyl alcohol, polyacrylonitrile, chitosan, polyurethane or polyethylene phthalate fibres, fibres formed from a blend of polymers, and mixtures thereof.

6. The product according to claim 1, wherein the fibres are chosen from polyamide fibres, cellulose fibres, polyethylene fibres, and mixtures thereof.

7. The product according to claim 1, wherein the oily phase of the W/O emulsion is in the form of a soft paste at room temperature.

8. The product according to claim 7, wherein the soft paste is obtained by heating the wax(es) and any other fatty substances of the oily phase, followed by blending while cooling to room temperature.

9. The product according to claim 1, wherein the W/O emulsion comprises at least one emulsifying surfactant chosen from the group consisting of oxyethylenated and/or oxypropylenated polydimethylsiloxanes, oxyethylenated and/or oxypropylenated C₁₀-C₂₂-alkyl-polydimethylsiloxanes, and oxyethylenated and/or oxypropylenated polydimethylsiloxanes having glucoside groups, and mixtures thereof.

10. The product according to claim 9, wherein the amount of emulsifying surfactant ranges from 0.02% to 5% by weight relative to the total weight of the W/O emulsion.

11. The product according to claim 1, wherein the wax of the W/O emulsion is chosen from carnauba wax, polyethylene waxes with a starting melting point of greater than 50° C., microcrystalline waxes with a starting melting point of greater than 50° C., and mixtures thereof.

12. The product according to claim 1, wherein the amount of wax(es) in the W/O emulsion ranges from 4% to 30% by weight relative to the total weight of the emulsion.

13. The product according to claim 1, wherein the W/O emulsion has an amount of oily phase ranging from 15% to 70% by weight relative to the total weight of the emulsion.

14. The product according to claim 1, wherein the first composition is in the form of a cream.

15. The product according to claim 1, wherein the second composition comprises an amount of oil representing at least 20% by weight relative to the total weight of the said composition.

16. The product according to claim 1, wherein the first composition comprises less than 0.2% of pigments, and wherein the second composition comprises at least 1% of pigments.

17. The product according to claim 1, wherein said product constitutes a foundation, a makeup rouge, an eye shadow, a concealer product, a blusher, a lipstick, a lip balm, a lip gloss, a lip pencil, an eye pencil, a mascara, an eye liner, a skincare product, a body makeup product or a skin colouring product.

18. The product according to claim 1, wherein the second composition is in the form of a lipstick in stick form, a cast foundation, a cast blusher or a liquid lip gloss.

19. A kit comprising the product of claim 1 and an applicator, wherein the first and second compositions are packaged separately.

20. A process comprising successively applying to the skin, the lips and/or the integuments the first and second compositions of the product according to claim 1.
